(19) United States
(12) Patent Application Publication
(10) Pub. No.: US 2025/0248771 A1
(43) Pub. Date: Aug. 7, 2025

(54) SYSTEMS AND METHODS FOR FACILITATING MEDICAL PROCEDURES WITH PRESENTATION OF A 3D POINT CLOUD

(71) Applicant: Intuitive Surgical Operations, Inc., Sunnyvale, CA (US)

(72) Inventors: Anthony M. Jarc, Johns Creek, GA (US); Omid Mohareri, San Francisco, CA (US); Richard Mahoney, Los Altos, CA (US)

(21) Appl. No.: 18/855,433

(22) PCT Filed: Apr. 24, 2023
(86) PCT No.: PCT/US2023/019623

§ 371 Date: Oct. 9, 2024

Related U.S. Application Data
(60) Provisional application No. 63/334,970, filed on Apr. 26, 2022.

(51) Int. Cl. A61B 34/00; A61B 34/10; A61B 34/20; A61B 90/00; G06T 15/20; G06T 19/00; G16H 70/20

(52) U.S. Cl. CPC A61B 34/25; G06T 15/205; G06T 19/006; A61B 2034/101; A61B 2034/2065; A61B 2034/256; A61B 2090/367; G06T 2210/41; G06T 2210/56; G06T 2219/004; G16H 70/20

(57) ABSTRACT

A medical procedure visualization system may be configured to access depth data captured by one or more depth capture devices during a medical procedure. The depth data may be representative of practitioner activity associated with the medical procedure. As such, the medical procedure visualization system may be further configured to render, based on the depth data, extended reality content for presentation to a user. The extended reality content may include a 3D point cloud depicting the practitioner activity associated with the medical procedure. Corresponding methods and systems are also disclosed.

### RELATED APPLICATIONS

The present application claims priority to U.S. Provisional Patent Application No. 63/334,970, filed Apr. 26, 2022, the contents of which is hereby incorporated by reference in its entirety.

### BACKGROUND INFORMATION

It may be useful to capture and record events occurring during a medical procedure (e.g., a surgical procedure, a diagnostic or exploratory procedure, a simulated training procedure, etc.) for a variety of purposes. For example, various types of records captured during a medical procedure (e.g., including video data, image data, sensor data, etc.) may be useful during the procedure to give visibility and context to practitioners (e.g., surgeons, physicians, nurses, assistants, trainees, etc.) observing or performing the procedure. In some examples, this may include practitioners who are located at the site of the medical procedure or at a remote site, as well as practitioners observing or participating with the medical procedure intraoperatively (i.e., in real time) and those observing the procedure postoperatively (i.e. after the fact) for training, outcome review and analysis, and/or other purposes.

One useful type of record for these and other use cases is graphical content (e.g., image and/or video content) that depicts the area where a medical procedure is performed. For instance, if a human body is being surgically operated on, video captured looking down onto the operating table (e.g., for an open surgery) or endoscopic video captured inside the body (e.g., for a non-invasive surgery) may be captured during the procedure and distributed to serve any of the use cases described above. Certain insights related to a medical procedure may not, however, be fully captured just from image or video content depicting the body receiving the operation. For example, activity occurring throughout the operating room (e.g., what practitioners in the operating room are doing at different times during the medical procedure, etc.) may be of interest, yet many details along these lines would not be reflected in endoscopic or other content that is focused only or primarily on the patient on which the medical procedure is performed.

### SUMMARY

Systems and methods for facilitating medical procedures with presentation of a 3D point cloud are described herein. For instance, one embodiment of a medical procedure visualization system configured to facilitate medical procedures in ways described herein includes one or more processors and memory storing executable instructions that, when executed by the one or more processors, cause the system to perform certain operations. For example, the operations may include accessing depth data captured by one or more depth capture devices during a medical procedure, the depth data representative of practitioner activity associated with the medical procedure. The operations may further include rendering, based on the depth data, extended reality content for presentation to a user, where the extended reality content includes a 3D point cloud depicting the practitioner activity associated with the medical procedure.

An example method embodiment may be performed by a medical procedure visualization system such as described above. For example, the method may include accessing depth data captured by one or more depth capture devices during a medical procedure, the depth data representative of practitioner activity associated with the medical procedure. The method may further include rendering, based on the depth data, extended reality content for presentation to a user, where the extended reality content includes a 3D point cloud depicting the practitioner activity associated with the medical procedure.

Yet another example embodiment may be implemented by a non-transitory, computer-readable medium storing instructions that, when executed, direct a processor of a computing device to perform operations of the method embodiment described above and/or any other operations described herein.

### BRIEF DESCRIPTION OF THE DRAWINGS

The accompanying drawings illustrate various embodiments and are a part of the specification. The illustrated embodiments are merely examples and do not limit the scope of the disclosure. Throughout the drawings, identical or similar reference numbers designate identical or similar elements.

FIG. 1 shows an illustrative medical procedure visualization system for facilitating medical procedures with presentation of 3D point clouds in accordance with principles described herein.

FIG. 2 shows an illustrative method for facilitating medical procedures with presentation of 3D point clouds in accordance with principles described herein.

FIG. 3 shows an illustrative computer-assisted medical system in accordance with principles described herein.

FIG. 4 shows an illustrative configuration in which one or more implementations of the medical procedure visualization system of FIG. 1 may operate in accordance with principles described herein.

FIG. 5A shows illustrative types of content that may be included within extended reality content in accordance with principles described herein.

FIG. 5B shows illustrative instances of extended reality content that include different content types in accordance with principles described herein.

FIG. 5C shows illustrative types of content that may be linked to and/or presented in connection with extended reality content in accordance with principles described herein

FIG. 6 shows an illustrative session in which a user experiences a medical procedure as facilitated by a presentation of a 3D point cloud in accordance with principles described herein.

FIG. 7 shows illustrative extended reality content that includes a 3D point cloud in accordance with principles described herein.

FIG. 8 shows illustrative viewpoints from which extended reality content may be rendered in accordance with principles described herein.

FIG. 9 shows a plurality of illustrative 3D point clouds that are included in an illustrative embodiment of extended reality content in accordance with principles described herein.

FIG. 10 shows an illustrative 3D point cloud library used to generate illustrative extended reality content in accordance with principles described herein.

FIG. 11 shows an illustrative computing system in accordance with principles described herein.

### DETAILED DESCRIPTION

Systems and methods for facilitating medical procedures with a presentation of a 3D point cloud are described herein. Various individuals performing, preparing for, learning about, or otherwise having interest in a medical procedure such as a surgery or other such medical procedure (e.g., an exploratory imaging operation, a mock medical procedure used for training purposes, etc.) may benefit from viewing a record of the procedure. For example, the record may be a video or other visualization of the procedure that is viewed either in real time (i.e., intraoperatively) or after the fact (i.e., postoperatively) for any of the purposes described herein. As mentioned above, while visualizations indicative of the area of the body being operated on may be helpful in recreating and/or providing understanding about a given medical procedure, these types of content may not tell the whole story of the medical procedure. For example, to gain a full and robust understanding of a medical procedure, it may be important to understand various aspects of activities performed by practitioners throughout the procedure. This practitioner activity may include, for instance, what tasks various individuals on the medical team are engaged in at various times during the procedure, what locations the individuals are located in (or moving toward) in response to certain events that occur during the procedure, and so forth.

To facilitate users in gaining this full understanding of a medical procedure, including practitioner activity of various people assisting in the performance of the medical procedure, medical procedure visualization systems described herein provide visualizations (e.g., extended reality content, etc.) that are configured to inform users (e.g., viewers of the visualizations) about practitioner activity during the medical procedure, as well as other events that may be occurring at the site of the medical procedure (e.g., within the operating room or other space in which the procedure is performed).

Surgery replay systems that have been deployed to capture and provide these types of insights about practitioner activity at the medical procedure site may be useful while still leaving ample room for improvement. For example, conventional surgery replay systems attempting to facilitate medical procedures with real-time or time-shifted representations of practitioner activity have typically involved 2D video that suffers from limited flexibility (e.g., not providing users an ability to view from different perspectives, etc.) or 3D modeling that suffers from heavy processing requirements and may appear unrealistic in ways that can be distracting to users. More specifically, current modeling technology is not generally advanced enough to provide volumetric models that are extremely lifelike and realistic, especially in applications where the content is being generated and viewed in real time. Moreover, conventional approaches also must address that sensitive content present at the site of the medical procedure (e.g., the identities of various practitioners, patient confidential information on charts or other documents in view within the operating room, etc.) may be readily visible absent deidentification efforts to sterilize the 2D video and/or 3D models of the sensitive content.

To address these and/or other shortcomings of conventional systems, systems and methods described herein are configured to facilitate medical procedures by way of the presentation of 3D point clouds that depict the practitioner activity associated with the medical procedure. This novel approach accounts for the fact that many users who view surgery replay content (e.g., experienced surgeons and other practitioners) often have more knowledge and understanding about the innerworkings of medical procedures than any surgery replay or 3D modeling algorithm is likely to be able to provide. Accordingly, systems and methods described herein may leverage the knowledge of users by presenting content that is closely based on raw captured data rather than being more highly processed interpretations of the captured data (e.g., as may be the case for volumetric models and/or other representations relied on by certain conventional surgery replay systems).

More particularly, medical procedure visualization systems described herein may facilitate a medical procedure (e.g., intraoperatively for remote proctoring or telesurgery, post-operatively for assessment or training purposes, etc.) by (1) accessing depth data, captured during the medical procedure, that represents practitioner activity associated with the medical procedure; and (2) rendering, for presentation to a user and based on the depth data, extended reality content (e.g., augmented or virtual reality content) that includes a 3D point cloud depicting the practitioner activity associated with the medical procedure. In this way, systems described herein may be configured to deliver extended reality content that can be generated with relatively low processing requirements while still having the customizability and viewing flexibility of 3D models (e.g., allowing users to observe the replay from viewpoints at arbitrary locations selected by the users, etc.). Extended reality content described herein may depict captured depth data representative of practitioner activity during the medical procedure in the raw form of a 3D point cloud, rather than (or in addition to) in the processed form of a volumetric mesh or voxelized 3D model. Not only does this ease the amount of processing that needs to be performed to generate the replay presentation, but the inherent nature of 3D point clouds also provides automatic deidentification of sensitive content and may help viewers focus on the most salient aspects of the presentation (e.g., rather than being distracted by 3D models that attempt to imitate the real world but are not quite lifelike enough to allow the user to feel immersed in a real environment).

Aspects of this disclosure are described in reference to computer-assisted medical systems and devices, which may include systems and devices that are manually manipulated, remote-controlled, semi-autonomous, autonomous, etc.; and which may include systems and devices that are robotic or non-robotic, teleoperated or non-teleoperated, and so forth. Certain aspects of this disclosure are described in terms of an implementation using a surgical system, such as the da Vinci® Surgical System commercialized by Intuitive Surgical, Inc. of Sunnyvale, California. However, inventive aspects disclosed herein may be embodied and implemented in various other ways. Implementations on da Vinci® Surgical Systems are merely illustrative and are not to be considered as limiting the scope of the inventive aspects disclosed herein. For example, techniques described with reference to surgical instruments and surgical methods may be used in other contexts. Thus, the instruments, systems, and methods described herein may be used for humans, animals, portions of human or animal anatomy, industrial systems, general robotic, or teleoperational systems. As further examples, the instruments, systems, and methods described herein may be used for medical purposes such as human or animal surgical procedures, sensing or manipulating tissue or non-tissue work pieces, cosmetic improvements, imaging of human or animal anatomy, gathering data from human or animal anatomy, setting up or taking down systems, training medical or non-medical personnel, and so forth. Additional example applications include use for procedures on tissue removed from human or animal anatomies (without return to a human or animal anatomy) and for procedures on human or animal cadavers. Further, these techniques can also be used for medical treatment or diagnosis procedures that include, or do not include, surgical aspects. Accordingly, the principles described herein have broad applicability to many

types of applications and use cases. The following description will often present a particular type of use case for convenience of description. Specifically, examples involving minimally-invasive surgical procedures performed on human patients using a computer-assisted (e.g., robotically-controlled) medical system such as a da Vinci® Surgical System will be described throughout this disclosure. It will be understood, however, that systems and methods described and illustrated herein may apply to, relate to, or be implemented with respect to various surgical and non-surgical medical procedures and/or operations (e.g., diagnostic procedures, treatment procedures, training procedures, etc.) for which medical personnel may utilize extended reality technologies such as, for example, virtual or augmented reality presentation devices. While such medical contexts are used as examples, it will be appreciated that the principles described herein may find significant applicability to various other types of contexts, scenarios, and applications.

Various benefits may be provided by systems and methods described herein for facilitating medical procedures with presentation of a 3D point cloud. As has been mentioned, for example, efficient and effective replay of practitioner activity may be provided to various users for various purposes including to train and educate practitioners on medical procedures generally, to help practitioners review and prepare for a particular upcoming procedure, to support intraoperative surgical assistance by remote practitioners (e.g., for telesurgery, proctoring, etc.), to postoperatively debrief and analyze the performance of a particular medical procedure, and so forth. By providing efficient and effective replay of medical procedures in the ways described herein, systems and methods described herein avoid many of the issues described above in relation to conventional surgery replay systems, including lack of viewpoint flexibility, rendering distracting or technically deficient (e.g., delayed, lagging, etc.) 3D models, and the like.

Moreover, other technical advantages may also arise, such as reduced computing resources needed to generate, store, transmit, and/or otherwise manage content based on 3D point clouds as compared to the computing resources need to manage fully volumetric 3D models and/or other more processed content. Additionally, as mentioned, the inherent nature of 3D point clouds is such that medical procedure visualization systems described herein may conveniently forego deidentification operations generally required for other types of content (e.g., to blur or remove sensitive patient or practitioner information that may be confidential and/or unlawful to publish to the audience to which the replay content is to be published).

Various specific embodiments will now be described in detail with reference to the figures. It will be understood that the specific embodiments described below are provided as non-limiting examples of how various novel and inventive principles may be applied in various situations. Additionally, it will be understood that other examples not explicitly described herein may also be captured by the scope of the claims set forth below. Systems and methods described herein may provide any of the benefits mentioned above, as well as various additional and/or alternative benefits that will be described and/or made apparent below.

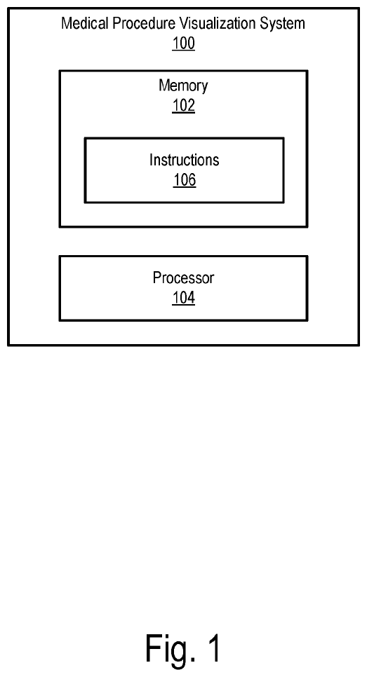

FIG. 1 shows an illustrative medical procedure visualization system 100 (“system 100”) for facilitating medical procedures with presentation of 3D point clouds in accordance with principles described herein. In certain embodiments, system 100 may be implemented by computer resources (e.g., processors, memory devices, storage devices, etc.) included within a computer-assisted medical system, depth data capture system, extended reality presentation device, and/or other system or device that is described herein. In other embodiments, system 100 may be implemented by computing resources of a standalone device or by any other suitable computing resources as may serve a particular implementation.

As shown, system 100 may include, without limitation, a memory 102 and a processor 104 selectively and communicatively coupled to one another. Memory 102 and processor 104 may each include or be implemented by computer hardware that is configured to store and/or process computer instructions (e.g., software, firmware, etc.). Various other components of computer hardware and/or software not explicitly shown in FIG. 1 may also be included within system 100. In some examples, memory 102 and processor 104 may be distributed between multiple devices and/or multiple locations as may serve a particular implementation.

Memory 102 may store and/or otherwise maintain executable data used by processor 104 to perform any of the functionality described herein. For example, memory 102 may store instructions 106 that may be executed by processor 104. Memory 102 may be implemented by one or more memory or storage devices, including any memory or storage devices described herein, that are configured to store data in a transitory or non-transitory manner. Instructions 106 may be executed by processor 104 to cause system 100 to perform any of the functionality described herein. Instructions 106 may be implemented by any suitable application, software, firmware, code, script, and/or other executable data instance. Additionally, memory 102 may also maintain any other data accessed, managed, used, and/or transmitted by processor 104 in a particular implementation.

Processor 104 may be implemented by one or more computer processing devices, including general purpose processors (e.g., central processing units (CPUs), graphics processing units (GPUs), microprocessors, etc.), special purpose processors (e.g., application specific integrated circuits (ASICs), field-programmable gate arrays (FPGAs), etc.), image signal processors, or the like. Using processor 104 (e.g., when processor 104 is directed to perform operations represented by instructions 106 stored in memory 102), system 100 may perform various functions associated with facilitating medical procedures with presentation of 3D point clouds in accordance with principles described herein.

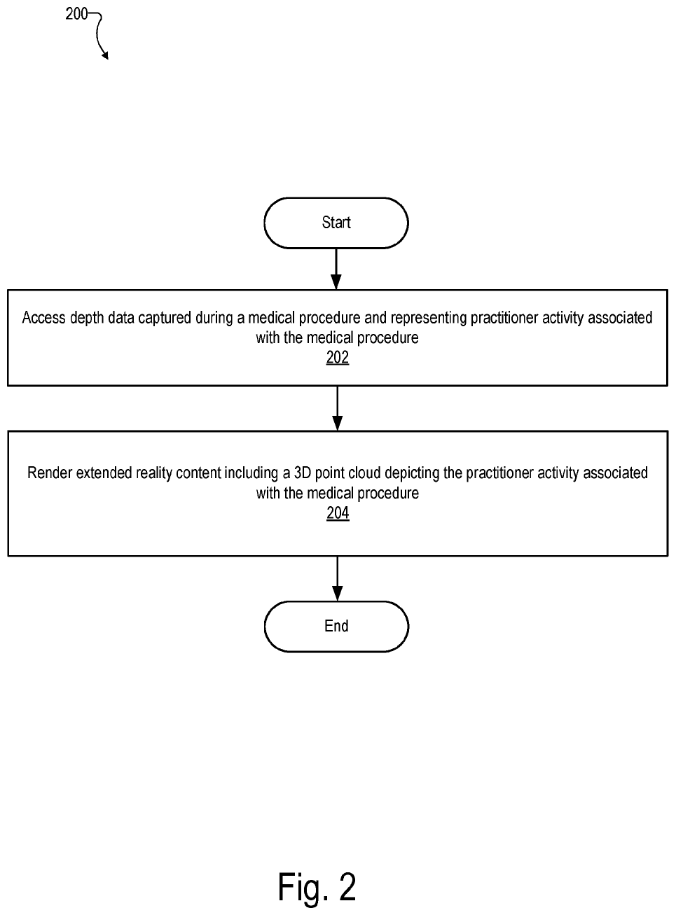

As one example of functionality that processor 104 may perform, FIG. 2 shows an illustrative method 200 for facilitating medical procedures with presentation of 3D point clouds that system 100 may perform in accordance with principles described herein. While FIG. 2 shows illustrative operations according to one embodiment, it will be understood that other embodiments may omit, add to, reorder, and/or modify any of the operations shown in FIG. 2. In some examples, multiple operations shown in FIG. 2 or described in relation to FIG. 2 may be performed concurrently (e.g., in parallel) with one another, rather than being performed sequentially as illustrated and/or described. One or more of the operations shown in FIG. 2 may be performed by a medical procedure visualization system such as system 100 or any implementation thereof.

In some examples, certain operations of FIG. 2 may be performed in real time so as to provide, receive, process, and/or use data described herein immediately as the data is generated, updated, changed, exchanged, or otherwise becomes available. Moreover, certain operations described herein may involve real-time data, real-time representations, real-time conditions, and/or other real-time circumstances. As used herein, “real time” will be understood to relate to data processing and/or other actions that are performed immediately, as well as conditions and/or circumstances that are accounted for as they exist in the moment when the processing or other actions are performed. For example, a real-time operation may refer to an operation that is performed immediately and without undue delay, even if it is not possible for there to be absolutely zero delay. Similarly, real-time data, real-time representations, real-time conditions, and so forth, will be understood to refer to data, representations, and conditions that relate to a present moment in time or a moment in time when decisions are being made and operations are being performed (e.g., even if after a short delay), such that the data, representations, conditions, and so forth are temporally relevant to the decisions being made and/or the operations being performed.

Each of operations 202 and 204 of method 200 will now be described in more detail as the operations may be performed by system 100 (e.g., by processor 104 as processor 104 executes instructions 106 stored in memory 102).

At operation 202, system 100 may access depth data captured by one or more depth capture devices during a medical procedure. For example, if the medical procedure is performed in an operating room, depth capture devices configured to capture depth data for people and objects in the room may be permanently installed or temporarily placed at various locations around the operating room. The depth capture devices may operate using any of various established depth detection or rangefinding techniques and/or principles such as, for instance, stereoscopic depth detection techniques, time-of-flight depth detection techniques, structured light depth detection techniques, or the like. The depth data captured by the depth capture devices may represent a large number of points in space where various surfaces (e.g., surfaces of people and/or objects within the operating room) have been detected based on the depth detection technology used.

Because the depth data is captured during the medical procedure (i.e., as the medical procedure is ongoing), the depth data accessed at operation 202 may be representative of practitioner activity associated with the medical procedure. The many points represented by the depth data may be represented as points in three-dimensional (3D) space (e.g., with x, y, and z coordinates with respect to a local coordinate system associated with the depth capture device and/or with respect to a world coordinate system associated with the entire operating room). Since these 3D points collectively form outlines of the objects (e.g., including people and/or inanimate objects) in the room without representing every possible surface point on the objects' surfaces, the points may be collectively referred to as a 3D point cloud and, when presented in relatively raw form, may give a viewer a good idea of what object in the room are, where the objects are located, how the objects are moving or behaving, and so forth, without revealing details about the texture of the object's surfaces (e.g., easily identifiable facial features of a human subject, textual or graphical content printed on walls or papers, etc.).

At operation 204, system 100 may render, based on the depth data, extended reality content for presentation to a user. For example, extended reality content rendered at operation 204 may be implemented by any type of content configured to exhibit any virtual objects (e.g., a 3D point cloud, a volumetric model, etc.) that are generated based on the depth data accessed at operation 202.

As one example, for instance, extended reality content rendered at operation 204 may be implemented as virtual reality content that presents a 3D point cloud in a virtual world distinct from an environment of the user as the user engages in a virtual reality experience based on the virtual reality content. In this case, for example, the user may be seated in a classroom remote from the operating room where the medical procedure is performed and may experience the medical procedure using a virtual reality headset that allows the user to view the 3D point cloud within a recreation of the operating room.

As another example, extended reality content rendered at operation 204 may be implemented as augmented reality content that presents the 3D point cloud as an augmentation overlaid onto an environment of the user as the user engages in an augmented reality experience based on the augmented reality content. In this case, for example, the user may be seated in an operating room (e.g., the same operating room in which the medical procedure was performed at an earlier time, a similar operating room to the one in which the medical procedure is being performed in real time, etc.) and may experience the medical procedure using an augmented reality headset that allows the user to view the 3D point cloud as if it is present in the room with the user (i.e., the user seeing mixed reality including real aspects of the room and the overlaid 3D point cloud).

One advantage of virtual and augmented reality described above is that the user may generally be able to interact with the extended reality environment being presented in some way. For instance, if wearing a headset, the user may look around in any direction and be presented with the virtual or augmented reality world in a natural and immersive way as if he or she is present in that world. Moreover, in some examples, the user may have the ability to move around within the world (e.g., by using movement controls or by physically walking about in his or her real environment). Extended reality content may therefore be a powerful way of experiencing an event such as a medical procedure, since the user may have the freedom to view the procedure from various perspectives using the extended reality technology.

In other examples, it will be understood that extended reality content may be implemented using other technologies that may not generally be considered to be virtual reality or augmented reality content and/or that may not provide the same level of movement and viewing flexibility. For instance, in certain implementations, a 3D point cloud may be integrated into video content that displays a background from a fixed perspective that the user is not able to change or interact with. Various examples of extended reality content and the viewpoints from which a user may view it will be described in more detail below.

The extended reality content rendered at operation 204 may include a 3D point cloud depicting the practitioner activity associated with the medical procedure. Accordingly, when the extended reality content is presented to a user, the user may, by observing the activities of the various practitioners helping to perform the medical procedure, be able to readily identify the roles (e.g., a surgeon, a scrub nurse, a first assistant, an anesthesiologist, etc.) and/or actions performed by the practitioners without necessarily being able to discern their personal identities by their facial features, clothing, hairstyle, or the like (since these details are inherently obscured by the nature of a textureless 3D point cloud).

Method 200 does not show an explicit step for presenting the extended reality content to the user, though this step will be understood to be performed either by system 100 or by a separate device or system that presents the content rendered by system 100 at operation 204. Accordingly, system 100 may be configured, in certain examples, to present the extended reality content rendered at operation 204 in an additional operation (not explicitly shown in method 200). In other examples, system 100 may be configured instead to provide the rendered extended reality content to a device (e.g., a head-mounted extended reality presentation device, a handheld extended reality presentation device, etc.) that is configured to present the content to the user (e.g., while being worn or otherwise used by the user in any of the ways described herein).

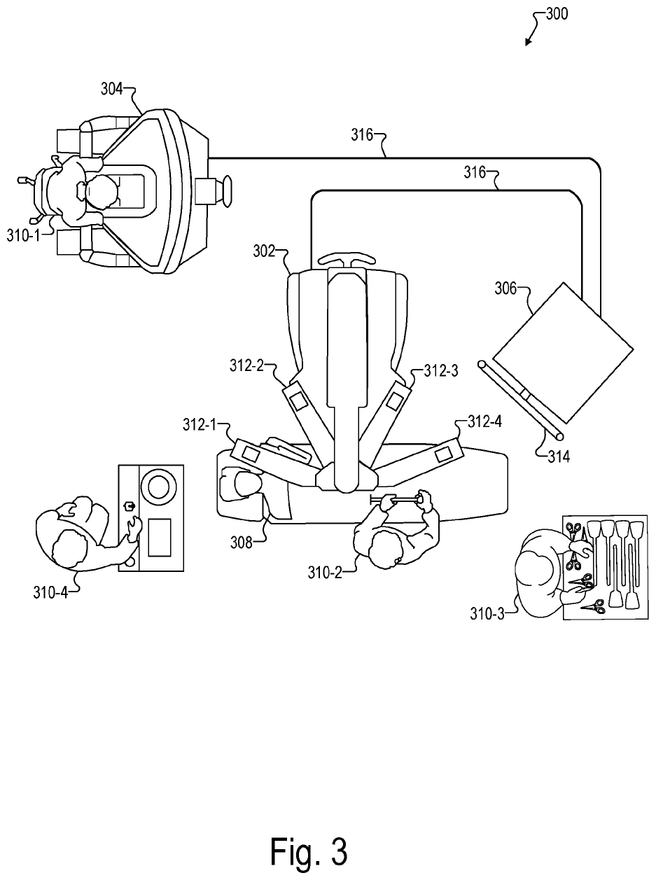

To set the stage for describing various illustrative aspects and examples of system 100 below, FIG. 3 will now be described. FIG. 3 shows an illustrative computer-assisted medical system 300 that may be used to perform various types of medical procedures including surgical and/or non-surgical procedures in accordance with principles described herein.

As shown, medical system 300 may include a manipulator assembly 302 (a manipulator cart is shown in FIG. 3), a user control system 304, and an auxiliary system 306, all of which are shown to be communicatively coupled to each other. Medical system 300 may be utilized by a medical team to perform a computer-assisted medical procedure or other similar operation on a body of a patient 308 or on any other body as may serve a particular implementation. As shown, the medical team may include a first practitioner 310-1 (such as a surgeon for a surgical procedure), a second practitioner 310-2 (such as a patient-side assistant), a third practitioner 310-3 (such as another assistant, a nurse, a trainee, etc.), and a fourth practitioner 310-4 (such as an anesthesiologist for a surgical procedure), all of whom may be collectively referred to as “practitioners 310,” and each of whom may control, interact with, or otherwise be a user of medical system 300 and, in some cases, a user of system 100 (in other examples, users of system 100 may be non-practitioners or others not involved in performing the medical procedure with practitioners 310). More, fewer, or alternative practitioners may be present during a medical procedure as may serve a particular implementation. For example, team composition for different medical procedures, or for non-medical procedures, may differ and include users with different roles.

While FIG. 3 illustrates an ongoing minimally invasive medical procedure such as a minimally invasive surgical procedure, it will be understood that medical system 300 (or a medical system replacing medical system 300 in FIG. 3) may similarly be used to perform open medical procedures or other types of operations. For example, operations such as exploratory imaging operations, mock medical procedures used for training purposes, and/or other operations may also be performed.

As shown in FIG. 3, manipulator assembly 302 may include one or more manipulator arms 312 (FIG. 3 shows manipulator assembly 302 as including a plurality of robotic manipulator arms 312 (e.g., arms 312-1 through 312-4)) to which one or more instruments may be coupled. The instruments may be used for a computer-assisted medical procedure on patient 308 (e.g., in a surgical example, by being at least partially inserted into patient 308 and manipulated within patient 308). While manipulator assembly 302 is depicted and described herein as including four manipulator arms 312, it will be recognized that manipulator assembly 302 may include a single manipulator arm 312 or any other number of manipulator arms as may serve a particular implementation. Additionally, it will be understood that, in some examples, one or more instruments may be partially or entirely manually controlled, such as by being handheld and controlled manually by a person. For instance, these partially or entirely manually controlled instruments may be used in conjunction with, or as an alternative to, computer-assisted instrumentation that is coupled to manipulator arms 312 shown in FIG. 3.

During the medical operation, user control system 304 may be configured to facilitate teleoperational control by practitioner 310-1 of manipulator arms 312 and instruments attached to manipulator arms 312. To this end, user control system 304 may provide practitioner 310-1 with imagery of an operational area associated with patient 308 as captured by an imaging device.

To facilitate control of instruments, user control system 304 may include a set of master controls. These master controls may be manipulated by practitioner 310-1 to control movement of the manipulator arms 312 and/or any instruments coupled to manipulator arms 312.

Auxiliary system 306 may include one or more computing devices configured to perform auxiliary functions in support of the medical procedure, such as providing insufflation, electrocautery energy, illumination or other energy for imaging devices, image processing, or coordinating components of medical system 300.

In some examples, auxiliary system 306 may be configured with a display monitor 314 configured to display one or more user interfaces, or graphical or textual information in support of the medical procedure. In some instances, display monitor 314 may be implemented by a touchscreen display and provide user input functionality. Extended reality content provided by a medical procedure visualization system such as system 100 may be similar, or differ from, content associated with display monitor 314 or one or more display devices in the operation area (not shown).

As will be described in more detail below, system 100 may be implemented within or may be operated in conjunction with medical system 300. For instance, in certain implementations, system 100 may be implemented entirely by one or more extended reality presentation devices associated with individual practitioners 310.

Manipulator assembly 302, user control system 304, and auxiliary system 306 may be communicatively coupled one to another in any suitable manner. For example, as shown in FIG. 3, manipulator assembly 302, user control system 304, and auxiliary system 306 may be communicatively coupled by way of control lines 316, which may represent any wired or wireless communication link as may serve a particular implementation. To this end, manipulator assembly 302, user control system 304, and auxiliary system 306 may each include one or more wired or wireless communication interfaces, such as one or more local area network interfaces, Wi-Fi network interfaces, cellular interfaces, and so forth.

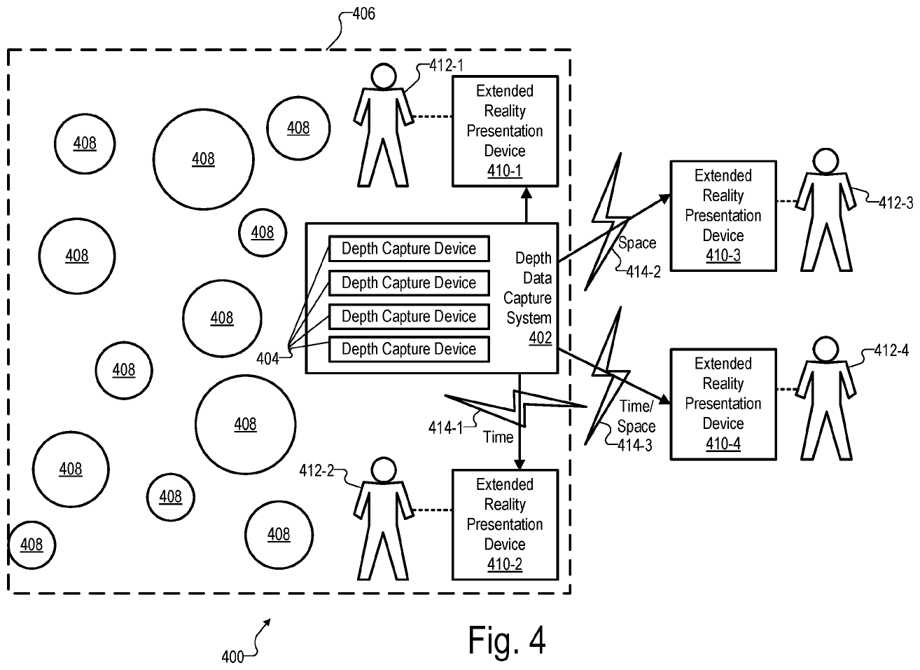

FIG. 4 shows an illustrative configuration 400 in which one or more implementations of system 100 may operate in accordance with principles described herein. Specifically, as shown and as will be described in more detail below, a depth capture system 402 that includes a plurality of depth capture devices 404 is configured to capture depth data at a site 406 where a medical procedure is being performed. The depth data captured by depth data capture system 402 may represent various objects 408 present at site 406 such that extended reality content representative of the objects 408 may be presented by any of various extended reality presentation devices 410 (e.g., devices 410-1 through 410-4) to thereby provide extended reality experiences for respective users 412 of the devices (i.e., users 412-1 through 412-4 of devices 410-1 through 410-4, respectively). Certain time and/or space discontinuities 414 (e.g., discontinuities 414-1 through 414-3) show that extended reality content may be presented in real-time or time-shifted ways locally at site 406 or remotely at other locations.

While configuration 400 illustrates several key concepts for generating and distributing extended reality content to thereby facilitate medical procedures with presentation of 3D point clouds as described herein, it will be understood that this configuration is illustrative only, and that certain configurations may include more or fewer elements than are illustrated in FIG. 4. As one example, one or more networks (e.g., local area networks, wide area networks, mobile carrier networks, the Internet, etc.) not explicitly shown in FIG. 4 may be used to transport data from one location to another (e.g., from depth data capture system 402 to one or more of extended reality presentation devices 410, etc.). Moreover, certain operations described as being performed by computing systems illustrated in FIG. 4 may, in certain implementations or configurations, be performed by computing resources of multi-access server computers (e.g., cloud servers, multi-access edge compute (MEC) servers, etc.) not explicitly shown in FIG. 4.

System 100 may be implemented by computing resources of any of the systems or devices shown in FIG. 4 or by computing resources of other systems or devices not explicitly illustrated. For example, in certain embodiments, instances of system 100 may be implemented by each device 410, which may access depth data from depth data capture system 402, render the extended reality content based on the depth data, and then present the extended reality content to the respective user 412 of the device. In other embodiments, an instance of system 100 may be implemented on depth data capture system 402. In this case, after accessing the depth data from depth capture devices 404 and rendering the extended reality content, the system 100 implementation may provide the rendered extended reality content in a standard video format to be presented by one or more of devices 410. In still other embodiments, an implementation of system 100 may be distributed across computing resources of both depth data capture system 402 and one or more of devices 410, and/or may be implemented or partially implemented by computing resources of other systems or devices not explicitly shown in FIG. 4. Each of the elements of FIG. 4 will now be described in more detail.

Depth data capture system 402 uses various depth capture devices 404 to capture depth data at and around site 406 of the medical procedure. To this end, depth data capture system 402 may include computing resources configured to consolidate and merge corresponding depth data captured by different depth capture devices positioned at different places at site 406. The depth data obtained, processed, and/or provided by depth data capture system 402 may be captured by the plurality of depth capture devices 404 shown to be part of depth data capture system 402. These depth capture devices may be arranged to have different vantage points at site 406 where the medical procedure is performed. For example, different depth capture devices 404 may be permanently fixed at the site (e.g., mounted in each corner of an operating room, etc.) or more temporarily set up (e.g., mounted on tripods, etc.) to capture a particular procedure. As will be described in more detail below, the depth data captured by depth capture devices 404 may provide sufficient information that the extended reality content may be rendered to show a 3D point cloud to the user from any of a variety of different viewpoints at site 406 where the medical procedure is performed.

Depth data capture system 402 and depth capture devices 404 may detect depth information about objects 408 at site 406 using any suitable depth capture technologies as may serve a particular implementation. For example, as mentioned above, time-of-flight depth detection techniques, stereoscopic depth detection techniques, techniques involving structured light, and/or other suitable techniques may be used.

In FIG. 4, site 406 is shown to represent the general area where the medical procedure is performed. For example, along with including an operating table on which the patient rests during the procedure, site 406 may also include various other people and objects that are relevant to the medical procedure and that may be important to observe to gain a thorough understanding of the medical procedure. In some examples, site 406 may represent a full or partial operating room in which the medical procedure is performed. Other adjoining rooms related to the operating room (e.g., a washroom, an observation theater, etc.) may also be included as part of site 406 in certain examples. In certain cases, a medical procedure may be performed in a location other than a dedicated operating room, such as in a doctor's office or clinic, in a medical tent during battle conditions, or the like. In these examples, site 406 would similarly represent the room or the area immediately surrounding the patient and the performance of the medical procedure.

While site 406 may be understood to encompass the entire room around where the medical procedure is performed in the example of FIG. 4, it will also be understood that, in certain examples, the site for which depth data is captured and for which extended reality content is rendered may be smaller in scope. For example, rather than showing practitioner activity throughout the entire operating room during a medical procedure, certain implementations may focus only on the operating table and the body being operated on, such that the practitioner activity exhibited by the 3D point cloud may focus less on where practitioners are moving within the room and more on whether practitioners around the operating table are efficiently working together (e.g., whether arms of different practitioners are crossing, whether every practitioner has space to stand and perform his or her role at the operating table, etc.).

Objects 408 may represent any objects or other visual content or imagery that may be present at site 406 to be captured by depth data capture system 402. For instance, in the example where site 406 includes a surgical operating room where a surgical medical procedure is being performed, objects 408 may represent an operating table upon which a body rests while the medical procedure is performed, various aspects of computer-assisted medical system 300 described above (e.g., manipulator assembly 302, user control system 304, auxiliary system 306, etc.), various practitioners (e.g., practitioners 310) moving about site 406 as the medical procedure is performed, and any other types of people or objects present in the operating room.

As mentioned above, certain objects at the site of a medical procedure may reveal sensitive information. For example, patient charts present in the room may reveal personal medical information about the patient, signs on the walls may reveal the identity of the hospital where the medical procedure is being performed, facial and other body features of practitioners performing the medical procedure may reveal the identities of the practitioners, and so forth. Depending on how the rendered extended reality content is to be used, it may be undesirable for some or all of these types of sensitive information to be identifiable within extended reality content that is generated representative of the medical procedure. Accordingly, 3D point clouds may be an advantageous way to represent objects 408 that would otherwise reveal sensitive information since these types of sensitive information are completely or largely concealed or obscured when the objects 408 are presented as textureless 3D point clouds rather than as video images or volumetric models.

As shown by arrows extending from depth data capture system 402 to extended reality presentation devices 410, raw depth data, fully rendered extended reality content, or other representations of the depth data captured at site 406 may be provided to extended reality presentation devices 410 for presentation to users 412. For example, as will be described and illustrated in more detail below, the extended reality content may comprise video content (e.g., moving 3D point clouds, moving volumetric models, etc.) textual content, graphical content (e.g., sensor images, symbols, drawings, graphs, etc.), and/or any combination of any of these or other types of content. Along with 3D point cloud of objects 408 at site 406, extended reality content presented by extended reality presentation devices 410 may also feature one or more external or internal views of the body receiving the medical procedure. Such views may be captured preoperatively or intraoperatively, and may be captured by any appropriate imaging device such as a camera for capturing visible light or non-visible light, an endoscope, an ultrasound module, a florescence imaging module, a fluoroscopic imaging module, a microphone to capture audio within site 406, or the like. In certain examples, augmentations displayed as part of the extended reality content may depict a model that has been generated based on preoperative data or that is generated and/or updated based on intraoperative data.

Each of devices 410 may be implemented by computer hardware and software (e.g., a processor, a memory storing instructions, a communications interface, etc.) of any suitable device that is configured to be used by one of users 412 to present extended reality content in ways directed by the user (e.g., based on a region of the world to which user 412 directs his or her viewpoint, etc.). In some examples, devices 410 may be implemented by head-mounted display devices configured to present extended reality content in a field of view of a wearer of the device as the wearer controls a viewpoint using head motions and/or physical controls (for virtual reality) or relocation of his or her own body (for augmented reality). In various implementations, head-mounted display devices may be implemented by dedicated extended reality devices, by general purpose mobile devices (e.g., tablet computers, smartphones, etc.) that are worn in front of the eyes using a head-mounting apparatus, or by other types of display devices.

In other examples, devices 410 may be implemented by devices that are not worn on the head. For instance, devices 410 may be implemented by a handheld device (e.g., a mobile device such as a smartphone, tablet, etc.) that may be pointed in different directions and/or focused to different distances within an extended reality world or by other non-head-mounted devices that are capable of presenting extended reality content in various other suitable ways.

In certain augmented reality implementations, one or more of devices 410 may include a camera that captures a view of the physical environment surrounding the device 410 and its user 412, and that passes the view in real-time through to a display screen viewable by the user. Certain general-purpose mobile devices used to implement device 410 may operate in this manner, for example. In other types of implementations, a device 410 may include a see-through screen that allows light to pass through from the physical environment to reach the eyes of the user 412 to allow augmented content to be presented on the screen by being overlaid onto the view of the physical world viewable through the screen.

Various devices 410 with different users 412 are shown in FIG. 4 to illustrate a few of the many use cases that may be served by extended reality content rendered to include 3D point clouds described herein. A few non-limiting example use cases will now be described.

Device 410-1 operated by user 412-1 will be understood to represent a use case in which the rendering of the extended reality content is performed in real time during the medical procedure for presentation to user 412-1 as an intraoperative extended reality experience. As shown, user 412-1 may be present at site 406 (e.g., in or near the operating room where the medical procedure is being performed) so as to observe, evaluate, or participate in the medical procedure while experiencing the intraoperative extended reality experience.

Device 410-2 operated by user 412-2 will be understood to represent a use case in which the rendering of the extended reality content is performed after the medical procedure is complete for presentation to the user as a postoperative extended reality experience. Discontinuity 414-1 (labeled “Time”) is shown to represent a break in time from when the depth data is captured by depth data capture system 402 and when the depth data is presented to the user as part of the extended reality content. For example, user 412-2 may review the medical procedure and the role that user 412-2 played in performing the procedure after the procedure is complete. As another example, user 412-2 may review the medical procedure prior to performing a similar medical procedure at the same site 406 to get an idea of pitfalls and things that worked well during the medical procedure when it was performed previously.

Device 410-3 operated by user 412-3 will be understood to represent a use case, similar to the use case described above in relation to device 410-1, in which the rendering of the extended reality content is performed in real time during the medical procedure for presentation to user 412-3 as an intraoperative extended reality experience. However, unlike the first use case described above, the use case illustrated by device 410-3 is shown to be associated with a discontinuity 414-2 (labeled “Space”) that represents a break in spatial continuity from site 406 and the location of device 410-3 and user 412-3. In this example, system 100 may thus direct the presentation of the extended reality content to user 412-3 while user 412-3 is physically remote from site 406 where the medical procedure is performed. For example, user 412-3 may be a practitioner (e.g., a surgeon) who is performing, assisting with, proctoring, consulting on, or otherwise involved with the medical procedure from a location remote from site 406. User 412-3 could be, for instance, a surgeon controlling a manipulator assembly (e.g., manipulator assembly 302) at site 406 from a user control system (e.g., user control system 304) that is remote from site 406. This setup may be useful for a telesurgery or hybrid medical procedure in which the surgeon is remote from the patient, a dual-console procedure in which a more experienced surgeon proctors or consults on the medical procedure from a remote user control system, a training scenario, or the like. In any of these examples, the extended reality content may provide the remote user 412-3 with situational awareness of where various people and objects are situated in the room while the medical procedure is ongoing, giving remote user 412-3 a sense of being present at site 406 even though he or she is not physical present (and may actually be alone in a room).

Device 410-4 operated by user 412-4 will be understood to represent a use case, similar to the use case described above in relation to device 410-2, in which the rendering of the extended reality content is performed after the medical procedure is complete for presentation to user 412-4 as a postoperative extended reality experience. However, unlike the second use case described above, the use case illustrated by device 410-4 is shown to be associated with a discontinuity 414-3 (labeled Time/Space) that represents a break in both spatial and temporal continuity. For example, user 412-4 may represent a medical student or a trainee learning to perform a certain type of medical procedure and/or to use particular equipment such as medical system 300. In these examples, the user may learn from the medical procedure even though he or she is remote from site 406 and experiencing the medical procedure after the fact. As will be described in more detail below, a library of 3D point clouds and/or extended reality content may be managed and used for such educational and training purposes and/or for other purposes as may serve a particular implementation. For example, while studying and practicing to serve one of the roles in the medical procedure, user 412-4 may view and analyze what actions are taken by the practitioner in the same role during this medical procedure and other medical procedures for which extended reality content is available from the library.

As described above, extended reality content rendered by system 100 and presented by one of devices 410 may be implemented by virtual reality content that presents a 3D point cloud in a virtual world distinct from an environment of the user as the user engages in a virtual reality experience, augmented reality content that presents the 3D point cloud as an augmentation overlaid onto an environment of the user as the user engages in an augmented reality experience, or other suitable extended reality content that integrates one or more 3D point clouds and/or other object representations (e.g., volumetric models, 2D video or imagery, etc.) into a real or virtual world that the user is experiencing.

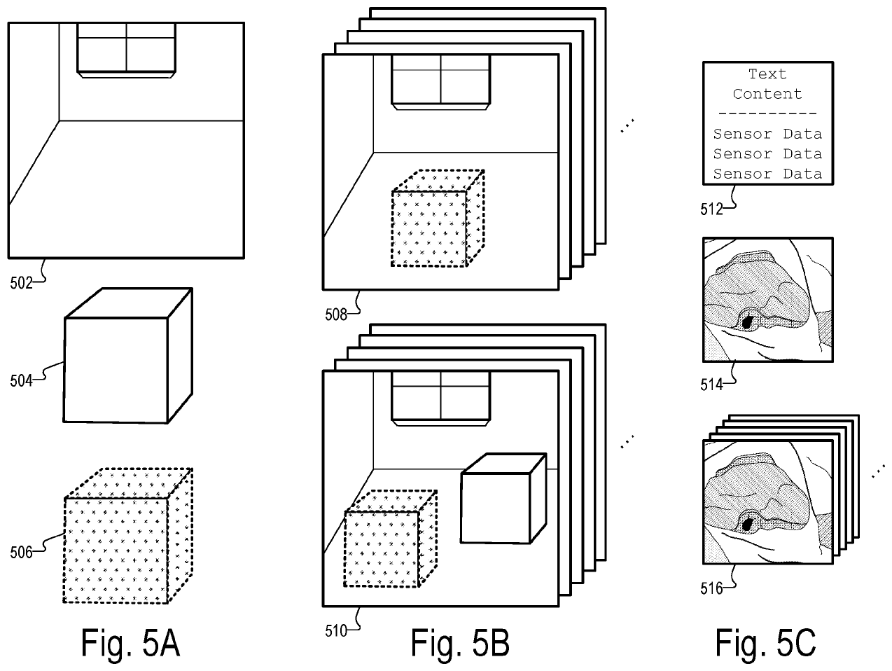

To illustrate some of the types of content that may be integrated into extended reality content in accordance with principles described herein, FIG. 5A shows an illustrative extended reality world 502, a volumetric model 504 of a particular object, and a 3D point cloud 506 of the same particular object.

Extended reality world 502 is drawn as a still image looking into a corner of a room. More particularly, the image shows portions of two walls, a floor area, and part of a window. In some examples, extended reality world 502 could be a still image or a live or prerecorded video feed of the world from a fixed vantage point of a mounted camera. In other examples, extended reality world 502 may represent a virtual world such as a computer-generated rendering of a real or virtual operating room. In such examples, rather than always being viewed from the static viewpoint illustrated in FIG. 5A, the extended reality world 502 may be viewed from various different vantage points based on which direction the viewer turns his or her head to look. In still other examples, extended reality world 502 may represent the real-world environment in which the user is actually located (e.g., passed through from a camera on the user's device 410 or viewed through a semi-transparent display as described above). Here again, in this example, the user would have complete control of his or her vantage point of extended reality world 502 based on where he or she is located within the world, which direction he or she chooses to look, and so forth.

Volumetric model 504 and 3D point cloud 506 each represent a same example object (e.g., a cube in this example) and are configured to be integrated into (e.g., added as virtual objects or augmentations to) extended reality world 502 to generate extended reality content that is presented to a user in any of the use cases described herein. While both volumetric model 504 and 3D point cloud 506 are illustrated in this example as basic cubic shapes for clarity of illustration, it will be understood that the objects represented by a volumetric model such as volumetric model 504 or by a 3D point cloud such as 3D point cloud 506 may include any of the types of objects 408 described above and/or any other suitable object that is to be presented within extended reality world 502. For example, volumetric models and/or 3D point clouds of practitioners, patients, equipment, and so forth captured at site 406 may all be represented as volumetric models and/or 3D point clouds for possible integration into an extended reality world such as extended reality world 502.

Volumetric model 504 is drawn as a white cube with solid faces to indicate that this model represents a 3D volumetric model generated based on depth data and texture data captured from a real-world object or generated for a purely virtual object. As such, it will be understood that a volumetric model of a human subject (e.g., a practitioner involved in performing the medical procedure) may appear relatively lifelike (e.g., due to being modeled based on depth data and texture data captured from various vantage points around the subject) but may also require significant amounts of processing that may be difficult or costly to accomplish in real time.

In contrast, 3D point cloud 506 is drawn as a cube formed from various points (i.e., dots) to indicate that this representation comprises a cloud of small points directly representing the depth data that has been captured for an object (e.g., without the processing to convert the points into a mesh, without a texture placed as a skin over the mesh, etc., as may be the case for volumetric model 504). While not explicitly shown in FIG. 5A, it will be understood that a 3D point cloud such as 3D point cloud 506 may be at least somewhat see-through (depending on the density of the points) and may be “textureless” in the sense that no image content (i.e., no texture layer) is applied to the captured depth data being shown by the points. Accordingly, while a volumetric model 504 of a human subject may appear more or less like the human subject looks in real life, a 3D point cloud of a human subject will appear as a human-shaped cloud of points.

As mentioned above, while the appearance of a 3D point cloud may inherently lack the realism that the appearance of a lifelike volumetric model may potentially have, 3D point clouds may be associated with their own advantages. For example, the 3D point cloud requires much less data processing to produce than a volumetric model (since it is essentially a rendering of the raw depth data that has been captured, rather than a complex model based on a combination of depth and texture data). As a result, the 3D point cloud may be more reliably presented without risk of lagging or processing errors. Moreover, at least for a person familiar with the basic protocols and roles of practitioners during a medical procedure, it may actually be preferable to see the raw data and know exactly what is being captured than to see a processed version of what has been captured that may be prone to error, delay, misinterpretation, and so forth. Additionally, as has been mentioned, the nature of a 3D point cloud such as 3D point cloud 506 is such that sensitive information associated with the object represented by the 3D point cloud is inherently obscured and deidentified, thereby avoiding another step that would be explicitly required for certain volumetric models 504 (as well as avoiding potential errors that could occur in the process of performing that step). For example, if sensitive textual information were printed on the cube object captured to generate volumetric model 504 and 3D point cloud 506, the textual information would be visible on volumetric model 504 (and would need to be removed as a separate step), while the textual information would not be represented by the cloud of 3D points making up 3D point cloud 506.

FIG. 5B shows illustrative instances of extended reality content (e.g., video content as illustrated by the series of frames depicted) that include different content types in accordance with principles described herein. More particularly, a first instance of extended reality content 508 shows a 3D point cloud (e.g., 3D point cloud 506) integrated into extended reality world 502 with no volumetric model shown to be included, while a second instance of extended reality content 510 shows an example of hybrid content that includes both a 3D point cloud (e.g., 3D point cloud 506) and a volumetric model (e.g., volumetric model 504) integrated into extended reality world 502. As extended reality world 502 may be understood to represent a site where a medical procedure is performed (e.g., site 406), the 3D point cloud and the volumetric model shown in extended reality content 510 will be understood to represent an object presented at the site where the medical procedure is performed.

In the example of extended reality content 508, system 100 may abstain from including object representations other than one or more 3D point clouds such as the 3D point cloud of the cube that is shown. In particular, as shown, the rendering of extended reality content 508 may include abstaining from including any volumetric model of any object. In this way, the benefits of 3D point cloud described above may benefit each different object. In some examples, the extended reality world may not be a room such as illustrated by extended reality world 502 but, rather, may be a black or white void or another suitable virtual space into which the 3D point cloud is integrated.

In contrast, in the hybrid content example of extended reality content 510, system 100 is shown to integrate a mix of object representation types including one or more 3D point clouds and one or more volumetric models. In this example, the 3D point clouds and volumetric models may be segmented into separate objects (e.g., separate practitioners; the patient and operating table; various components of medical system 300 such as manipulator assembly 302, user control system 304, auxiliary system 306; etc.). With objects segmented in this way, it may be possible for system 100 to render extended reality content 510 with each object as either a 3D point cloud (e.g., a sub-cloud of the original 3D point cloud) or as a volumetric model. The selection of which objects to render in which form may be performed in any suitable way based on any suitable criteria. For example, volumetric models may be used to represent inanimate objects (e.g., objects other than the practitioners and the patient), objects that are fixed in place and not moving, objects for which preexisting models (e.g., CAD models, etc.) happen to be available and do not need to be generated from scratch based on captured data, objects that are not associated with sensitive information, and/or other suitable subsets of the objects at the site. Conversely, 3D point clouds may be used to represent living beings (e.g., the practitioners and the patient), objects that dynamically move and need to be continuously updated as they do so, objects for which real-time captured data must be relied on since no preexisting models exist, objects that are associated with sensitive information that it is desirable to obscure for the extended reality content, and/or other suitable subset of objects at the site.

In some implementations, extended reality content such as extended reality content 508 or extended reality content 510 may be further augmented by additional content that may be of interest to the user viewing the medical procedure. To show examples of such additional content, FIG. 5C shows illustrative types of content that may be linked to and/or presented in connection with extended reality content. Specifically, as shown, text content 512 may include sensor data (e.g., data captured by body tracking sensors such as heart rate monitors and/or temperature monitors, locational data indicating where different objects are located, data reported by various types of instruments being used in the medical procedure, etc.) that may be of interest to a user experiencing extended reality content that depicts practitioner activity during a medical procedure. Image data 514 may depict endoscopic imagery or content resulting from other types of preoperative or intraoperative medical imaging (e.g., CT scans, ultrasound, fluorescence imaging, etc.). Similarly, video data 516 (differentiated from image data 514 by being depicted as a series of frames instead of a single image) may show a live or recorded feed of the same types of content described for image data 514.

The additional content may be presented together with the 3D point cloud in an extended reality environment in any manner as may serve a particular implementation. As one example, endoscopic video or other types of additional content may be presented on a screen within the virtual space where the 3D point cloud is presented (e.g., a virtual television on the wall of the operating room, etc.). As another example, the additional content may be anchored to the user's display (e.g., pinned to a corner of the field of view). In some implementations, it may be useful, when the user is experiencing extended reality content such as extended reality content 508 or extended reality content 510, to switch between the extended reality content and additional types of content such as shown in FIG. 5C, or to view such additional content presented together with the object representations (e.g., the 3D point clouds and/or volumetric models) in the extended reality world. For example, system 100 may link extended reality content such as extended reality content 508 or extended reality content 510 to an additional type of content depicting another aspect of the medical procedure occurring concurrently with the practitioner activity and may provide the user with an interactive presentation of the medical procedure that allows the user dynamic control to view the extended reality content and the additional type of content in any way the user may desire (e.g., to switch between the extended reality content and the additional content, to view both at once, to overlay one on the other, etc.).

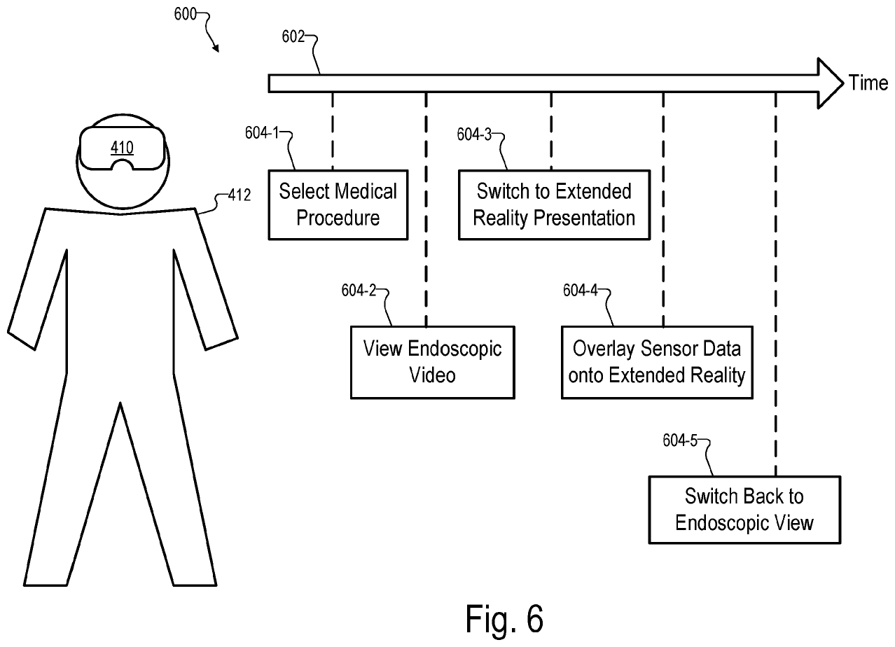

To illustrate an example, FIG. 6 shows an illustrative session 600 in which a user 412 experiences a medical procedure as facilitated by a presentation of 3D point clouds in accordance with principles described herein. Specifically, as shown, user 412 may use a head-mounted extended reality presentation device 410 to experience extended reality content as well as additional content that the user may desire to view together with or instead of the extended reality content at different points during the session.

A timeline 602 marked with various illustrative actions 604 (e.g., actions 604-1 through 604-5) indicates certain actions that user 412 may perform to control what he or she experiences (e.g., sees, hears, etc.) during session 600. As shown, for example, at action 604-1, user 412 may select a medical procedure to experience. This may involve choosing a medical procedure (or portion of a medical procedure) from a content library (e.g., a library such as will be described and illustrated in more detail below), connecting to a depth data capture system or other server configured to provide depth data or extended reality content including a 3D point cloud, or preparing to experience a particular medical procedure in another suitable manner.

Upon selecting the medical procedure, action 604-2 shows that user 412 may then view endoscopic video of the selected medical procedure. However, when a certain event occurs while watching the endoscopic video (e.g., a problem or anomaly is encountered that the surgical team must rush to address, a phase of the surgery is completed and a new phase is prepared for and commenced, etc.), action 604-3 shows that user 412 may switch the presentation to view the extended reality content including one or more 3D point clouds depicting the practitioner activity at the site of the medical procedure. If user 412 is preparing to perform a particular role in this type of medical procedure, for example, it may be of interest to user 412 to temporarily abstain from viewing from the endoscopic video when the event occurs (e.g., when the anomaly is encountered, etc.) and to instead watch how a practitioner with that particular role behaves in response to the event.

While experiencing the extended reality presentation after action 604-3, the user may wish to continue observing the practitioner activity depicted by the 3D point cloud while also getting visibility into other information such as the status of the patient. Accordingly, as shown at action 604-4, user 412 may overlay sensor data (e.g., sensor data indicative of the patient status, etc.) onto the extended reality content so as to be able to view both at the same time. Subsequently (e.g., when the anomaly has been addressed, when the new phase of the surgery has commenced, etc.), user 412 may desire to again view what is happening within the body and may therefore switch back to the endoscopic view at action 604-5.

In some examples, the switching between different types of presentations (e.g., the extended reality content, the endoscopic or other video view, the sensor data, etc.) may be performed automatically without the user being required to manually indicate the changes. For instance, if a notable increase in practitioner activity is detected, the 3D point cloud may automatically be switched over or overlaid onto whatever the user is currently viewing.

As used herein, a 3D point cloud generally refers to all the 3D points represented by all the depth data captured at a site of a particular medical procedure. In other words, various objects 408 (e.g., practitioners, medical systems, instruments, equipment, etc.) may all be represented as part of a single 3D point cloud that is presented to represent the objects 408 of a particular site 406. In some examples, however, it will be understood that certain objects (or parts of objects) of a set of objects at a site may be segmented (e.g., distinguished, separated, etc.) from other objects (or parts of objects) and treated like individual, discrete entities, rather than as composite parts of a single unified 3D point cloud. Such segmented portions of a 3D point cloud are referred to herein as discrete “sub-clouds”. To assist users in being able to distinguish between different objects, such as to track different practitioners moving about the site during the medical procedure and/or to easily tell the difference between practitioners and inanimate objects in the room, certain differentiable effects (e.g., colors, sounds, labels, etc.) may be added to various sub-clouds of the 3D point cloud.

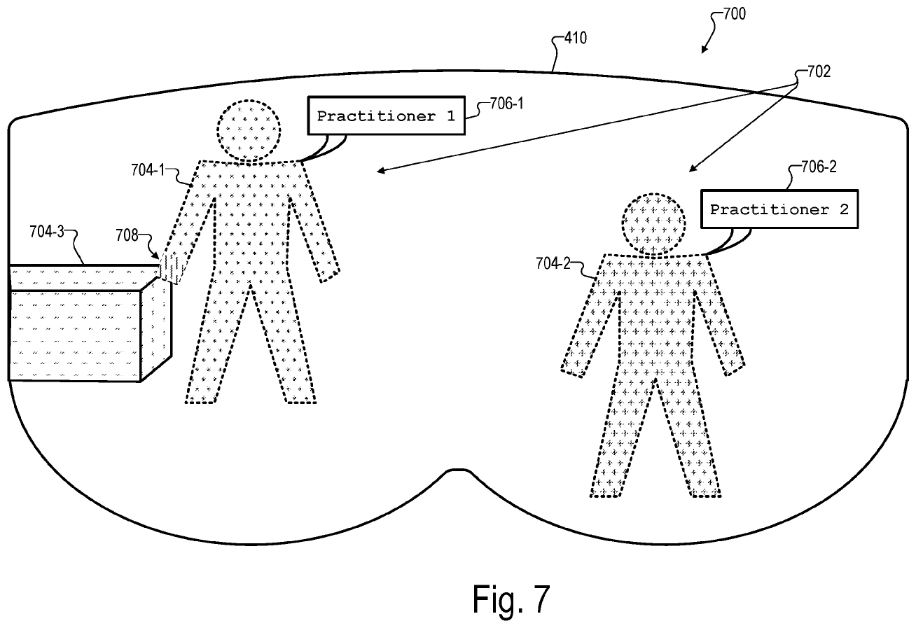

To illustrate, FIG. 7 shows example extended reality content 700 that features a 3D point cloud 702 comprising all the points of all the people/objects shown (e.g., all the depth data captured at a particular site during a particular medical procedure). As shown, 3D point cloud 702 includes a plurality of discrete sub-clouds 704 (e.g., sub-clouds 704-1 through 704-3) that are segmented to represent individual people and objects present at the site. As shown, extended reality content 700 is viewed by way of a device 410 that is illustrated in FIG. 7 (and other illustrations herein) roughly in the shape of a head-mounted display to suggest how certain implementations of device 410 may be worn and used to experience extended reality content 700. When rendering extended reality content 700 with 3D point cloud 702, system 100 may differentiate the different sub-clouds 704 and highlight various aspects of the practitioner activity associated with the medical procedure in various ways that will now be described.

As one example, 3D point cloud 702 shows that the rendering of extended reality content 700 by system 100 may include visually differentiating a first practitioner represented by 3D point cloud 702 from a second practitioner represented by 3D point cloud 702 by using a first color for points representing the first practitioner and using a second color different from the first color for points representing the second practitioner. In FIG. 7, different colors of points are represented by different point styles, including small circular points representing the first practitioner of sub-cloud 704-1, small ‘+’-shaped points representing the second practitioner of sub-cloud 704-2, and small ‘−’-shaped points representing the box-shaped object of sub-cloud 704-3. As shown by these different point styles, different colors may be used for each distinct, segmented object at the scene (e.g., each different person and thing shown) to help a user experiencing extended reality content 700 easily differentiate the distinct objects being represented.

In some examples, rather than using color to distinguish every individual object, colors (or other suitable ways of distinguishing sub-clouds) may instead be used to differentiate different classes or types of objects, to highlight specific objects, and so forth. For example, sub-clouds representing practitioners could be presented with points of a first color, a sub-cloud representing the patient on which the medical procedure is being performed could be presented with points of a second color, and inanimate (non-human) objects in the room such as a medical system and its various components could be presented with points of a third color. As another example, a particular practitioner (e.g., a practitioner playing a role of particular interest to the user experiencing the extended reality content) may be presented with points of a first color while all the other practitioners associated with the medical procedure could be presented with points of a second color.

As another example of how sub-clouds may be differentiated, 3D point cloud 702 shows that the rendering of extended reality content 700 by system 100 may include visually differentiating the first practitioner represented by 3D point cloud 702 from the second practitioner represented by 3D point cloud 702 by using a first label 706-1 (“Practitioner 1”) for the first practitioner and using a second label 706-2 (“Practitioner 2”), which is different from the first label, for the second practitioner. These labels may indicate the particular roles of the practitioners (e.g., “Surgeon”, “First Assistant”, “Scrub Nurse”, “Anesthesiologist”, etc.), the names or other identifying features of the people, or any other information as may serve a particular implementation. As shown, it may not be considered helpful in certain implementations to include a label for the inanimate object represented by sub-cloud 704-3, while in other implementations it will be understood that such objects may also have their own labels.

In some implementations, certain parts of an object (e.g., a portion of a sub-cloud representing that object) may be highlighted in comparison to other parts of the object. For instance, the rendering of extended reality content 700 by system 100 may include visually highlighting a first aspect of the 3D point cloud to emphasize the first aspect over a second aspect of the 3D point cloud by using a first color for points representing the first aspect and using a second color different from the first color for points representing the second aspect. The head or hands of one or more practitioners could be presented with points of a different color than the rest of their bodies, for example, or an instrument could be presented with points of a different color than the manipulator arms of a manipulator assembly system that controls the instrument (none of which is explicitly shown in FIG. 7).

Other examples of highlighting aspects of a 3D point cloud may also be implemented for situations, such as shown in FIG. 7, in which the 3D point cloud depicting the practitioner activity includes a plurality of discrete sub-clouds 704 representing individual people and objects associated with the medical procedure. For instance, 3D point cloud 702 shows that the rendering of extended reality content 700 by system 100 may include visually indicating interaction between different discrete sub-clouds 704-1 and 704-3 by using a first color for adjacent or overlapping points from different discrete sub-clouds and using a second color for other points not adjacent or overlapping with points from different discrete sub-cloud. Specifically, as shown by a different point style of a portion 708 of the sub-clouds at an interaction between the arm of the first practitioner and a corner of the object represented by sub-cloud 704-3, the interaction between these discrete sub-clouds 704 (e.g., where the distinct sub-clouds are adjacent or overlapping due to objects touching or nearly touching one another) may be highlighted using a different color than either of the sub-clouds 704. This may help the user better gauge depths and understand geometrical aspects of how objects are interacting in the absence of other visual cues such as shadows that might otherwise be relied on for object representations other than 3D point clouds.

In addition or as an alternative to being used to differentiate sub-clouds or object interactions, colored points in 3D point clouds being presented may be used to indicate other types of information as well. As one additional example, color (or similar visual effects such as shade or brightness) may be used to indicate time lapse as the 3D point cloud changes and objects represented by the 3D point cloud move in time. For instance, points representing the 3D point cloud at a present moment may be presented as a certain color, shade, and/or brightness while points of the 3D point cloud at previous moments (in the past) may be presented in different colors, shades, and/or brightnesses. In this way, an effect could be produced that would cause a “tail” behind a moving sub-cloud as the points occupied by the sub-cloud in the recent past would slowly fade in color or shade (e.g., from red to blue, from a bright shade of red to darker shades of red, etc.), or become darker or the like, until disappearing completely after a certain period of time. Such effects may make it easier for the user to visually perceive the movement and flow of various individual objects (e.g., sub-clouds) in the presentation.

In certain implementations, extended reality content 700 may be configured to not only include visual content but to further include sound and/or other types of content (e.g., haptic content, a time counter, etc.) configured to be presented together with 3D point cloud 702 depicting the practitioner activity associated with the medical procedure. For example, sound may be recorded during the medical procedure in certain examples or, in other examples, may be added to the extended reality content as an augmentation after the medical procedure is complete. Sound integrated into the extended reality content may provide context as to what practitioner activity is occurring, what the roles and responsibilities of different practitioners are, what the practitioners communicate amongst themselves during the medical procedure, and/or any other context as may serve a particular implementation.

As mentioned above in relation to depth data capture system 402 and depth capture devices 404, depth data may be captured by a plurality of depth capture devices arranged to have different vantage points at the site where the medical procedure is performed, and extended reality content may then be rendered, based on this depth data, to show a 3D point cloud to a user from a particular viewpoint at the site where the medical procedure is performed. The particular viewpoint from which the user experiences the 3D point cloud (and other aspects of the extended reality content such as volumetric models in a hybrid example such as described above) may be static or dynamic, predetermined or under control of the user, and/or otherwise flexibly set and/or modified as may serve a particular implementation.

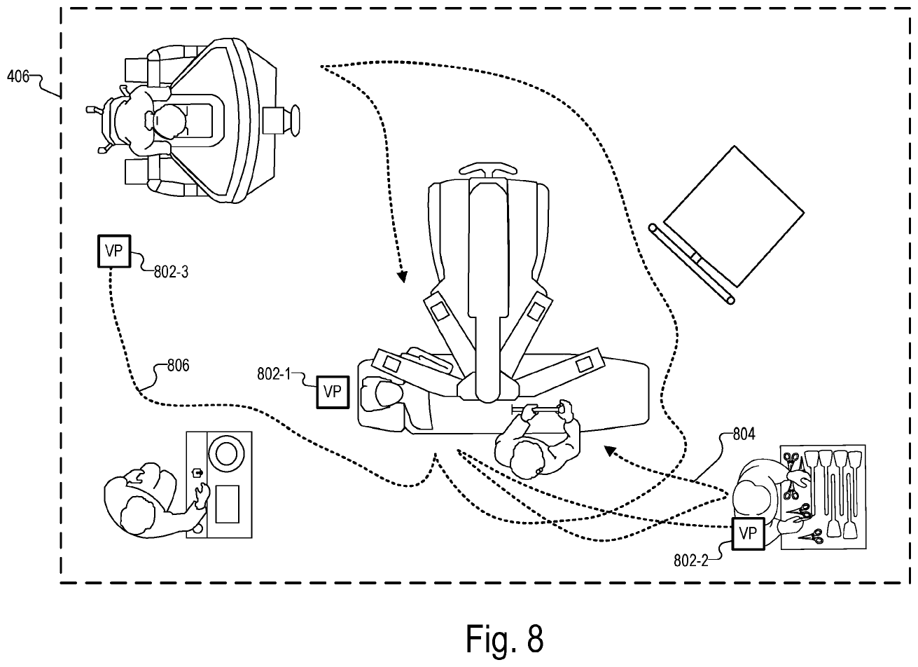

To illustrate, FIG. 8 shows several illustrative viewpoints 802 (e.g., viewpoints 802-1 through 802-3) that are drawn as boxes labeled “VP” and located at particular locations within an implementation of site 406 that includes various practitioners and components of the medical system 300 described in relation to FIG. 3.

More particularly, as shown in the example of viewpoint 802-1, the particular viewpoint from which the user experiences the medical procedure may be a static viewpoint tethered to a static location at the site during the medical procedure. For instance, as shown, the static location may be near the operating table (e.g., the focal point of the practitioner activity) so that the user may be able to see what is going on all around the patient throughout the medical procedure. The static viewpoint at which viewpoint 802-1 is located may be distinct from any of the different vantage points of the plurality of depth capture devices arranged at the site. As such, the 3D point cloud viewed from viewpoint 802-1 may utilize depth data consolidated from multiple depth capture devices rather than merely representing depth data captured by a single depth capture device that is located at a common point in space (or being otherwise aligned) with the static viewpoint.

As shown in the example of viewpoint 802-2, another implementation of the particular viewpoint may include a dynamic viewpoint tethered to a practitioner moving about at the site during the medical procedure. Specifically, as shown, viewpoint 802-2 may be anchored or tethered to a particular practitioner who, as indicated by a path 804 that the practitioner may follow in due course of time as he or she carries instruments to and from the operating table, may move around site 406 during the medical procedure. This type of viewpoint may be especially useful to a user attempting to study the activity of a particular role or practitioner since the user can effectively “follow” that practitioner throughout the entire performance of the medical procedure.

As shown in the example of viewpoint 802-3, another implementation of the particular viewpoint may be dynamically selected and changed by the user as the user experiences the extended reality content. In other words, the user may have the ability to freely move his or her viewpoint around site 406 throughout the medical procedure to observe whatever is of interest to the user, to study different aspects of the procedure during different viewings of the content, and so forth. To illustrate this, viewpoint 802-3 is shown to follow a path 806 that will be understood to represent the path that the user may choose to pursue over a period of time as the medical procedure is being performed.

In other examples, instead of the user himself or herself choosing path 806 and having freedom to move about site 406 at will, the particular viewpoint may be selected (e.g., by a machine learning process, by a human curator, or by way of another automatic or manual process) as part of a creation of a library of 3D point cloud data configured to demonstrate a variety of medical procedures including the medical procedure. For example, if the medical procedure is determined to be a good example of a particular type of procedure and selected for inclusion in a library used for training practitioners learning to participate in this type of procedure, a recommended path (e.g., path 806) may be designated and associated with the library entry to help ensure that the user experiences the most relevant aspects of the medical procedure from optimal locations at the site. This recommended path may be based on paths chosen by other users who have experienced the medical procedure (e.g., using machine learning or another automatic process to determine what the other users considered optimal) or may be explicitly designated by a curator (e.g., an expert who selected the medical procedure for inclusion in the library and who has specific aspects of the medical procedure in mind that should be emphasized to those who will experience the extended reality content). Examples of content libraries will be described in more detail below.

While most of the examples described above have involved extended reality content with a single 3D point cloud (possibly including multiple sub-clouds) representing a single medical procedure performed at a particular site, certain examples of extended reality content may involve more than one 3D point cloud concurrently representing more than one medical procedure performed at one or more sites. For example, along with performing the operations of method 200 described above, system 100 may be configured to further access additional depth data captured during an additional medical procedure distinct from the medical procedure, where the additional depth data is representative of practitioner activity associated with the additional medical procedure. The extended reality content rendered at operation 204 may then be further rendered, based on the additional depth data, to include an additional 3D point cloud depicting (simultaneously with the practitioner activity associated with the first medical procedure) the practitioner activity associated with the additional medical procedure. These 3D point clouds may be simultaneously presented in any suitable way. For instance, the 3D point clouds may be presented in a side-by-side manner (e.g., so that the 3D point clouds are separated but both depicted at the same time in the same space), in a direct overlay manner (e.g., so that the 3D point clouds are anchored at a common viewpoint and intersecting within the same space), in a multiplexed manner (e.g., so that the user can conveniently switch between viewing the different point clouds such as by pushing a button or moving a slider), or in any other manner as may serve a particular implementation.

The simultaneous exhibiting of multiple 3D point clouds associated with practitioner activity of multiple different medical procedures may be advantageous for comparing the medical procedures for a variety of purposes. As one example, extended reality content including multiple 3D point clouds such as described above may be used as training content configured to demonstrate optimal and suboptimal practitioner activity to the user for a medical procedure type (e.g., a specific type of surgery or other operation). For instance, the medical procedure and the additional medical procedure may both be of the same medical procedure type, the medical procedure may be included in the training content as an optimal instance of the medical procedure type, and the additional procedure may be included in the training content as a suboptimal instance of the medical procedure type. By viewing respective 3D point clouds for both the optimal and suboptimal instance of the medical procedure type in this extended reality content, the user may gain insight into practitioner-activity-related details that led to the optimal and suboptimal outcomes. For example, the user may note that the team responded to an anomaly more quickly or in a different way in the optimal medical procedure than did the team in the suboptimal medical procedure.

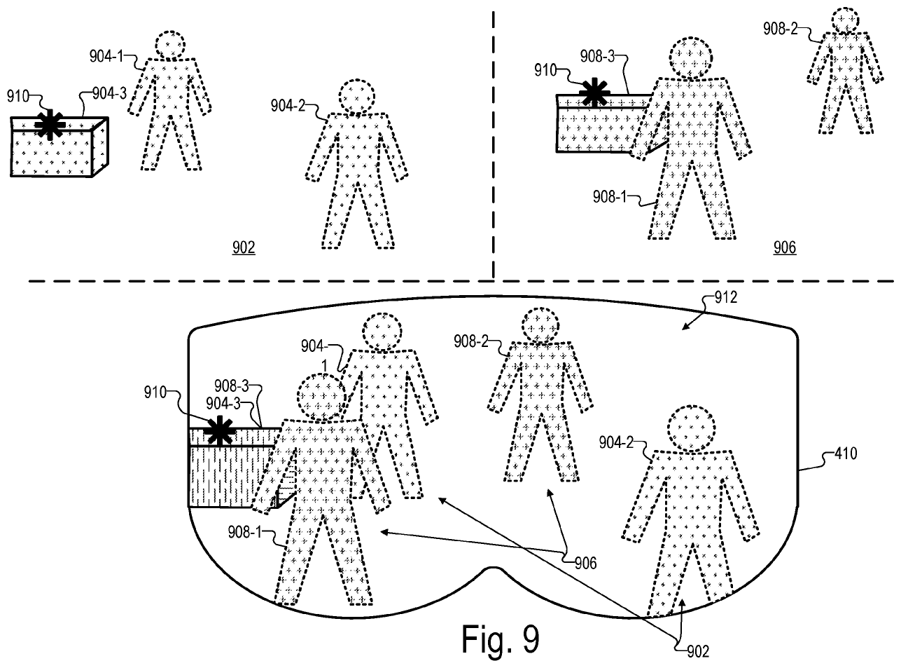

To illustrate some of these principles, FIG. 9 shows a plurality of illustrative 3D point clouds that are included in an illustrative embodiment of extended reality content in accordance with principles described herein. Specifically, as demarcated by dashed lines in the top-left quadrant of the figure, a 3D point cloud 902 is shown to include several sub-clouds 904 representing a first practitioner (sub-cloud 904-1), a second practitioner (sub-cloud 904-2), and a box-shaped inanimate object (sub-cloud 904-3) that may represent an operating table or other object at the site of this particular medical procedure. Similarly, as demarcated by dashed lines in the top-right quadrant of the figure, a 3D point cloud 906 is shown to include several sub-clouds 908 representing a first practitioner (sub-cloud 908-1), a second practitioner (sub-cloud 908-2), and a similar box-shaped inanimate object (sub-cloud 908-3) that may represent a similar operating table or other object that happens to also be at the site of this additional medical procedure. Assuming that the operating table represented by the inanimate objects of sub-clouds 904-3 (in 3D point cloud 902) and 908-3 (in 3D point cloud 906) are a focal point of the respective medical procedures being performed, an anchor point 910 is identified within each of these sub-clouds 904-3 and 908-3. For example, this anchor point may represent a particular corner of the tables, an entry point into the patient's body where instrumentation (e.g., an endoscope and/or manipulator instruments) are inserted to perform the medical procedure, or the like.

In the bottom half of FIG. 9, a similar implementation of a device 410 is shown to illustrate a presentation of extended reality content 912, which may incorporate both 3D point cloud 902 and 3D point cloud 906. Indeed, as illustrated, extended reality content 912 is rendered such that, when presented to the user in device 410, 3D point cloud 902 and additional 3D point cloud 906 are concurrently presented to the user in a shared virtual space (i.e., in the direct overlay manner mentioned above, as if all the sub-clouds 904-1 through 904-3 of 3D point cloud 902 share the same virtual world with all the sub-clouds 908-1 through 908-3 of 3D point cloud 906). To create this effect in a productive and intuitive way for the user to gain insight from the concurrent 3D point clouds, system 100 is shown to align the 3D point clouds within extended reality content 912 at the corresponding anchor points 910 common to the medical procedure and the additional medical procedure. In other words, as shown, anchor point 910 is aligned such that the operating tables represented respectively by sub-clouds 904-3 and 908-3 are almost entirely overlapping (and are illustrated to have a different point style than other sub-clouds presented in extended reality content 912) and the various practitioners from each of 3D point clouds 902 and 906 are presented concurrently at their respective locations with respect to anchor point 910.

In extended reality content implementations involving multiple 3D point clouds concurrently presented to the user in a shared virtual space, the extended reality content may be generated such that the first 3D point cloud is rendered with a first color and the additional 3D point cloud is rendered with a second color distinct from the first color. To illustrate, different point styles are shown in FIG. 9 for the sub-clouds 904 of 3D point cloud 902 and for the sub-clouds 908 of 3D point cloud 906. As mentioned, the operating table of sub-clouds 904-3 and 908-4 may appear to be yet another color due to the way that the sub-clouds proximate to anchor point 910 largely or entirely overlap. This is because point clouds tend to be translucent (e.g., partially transparent) according to the size and density of the points making up the point cloud.

Though this translucency is not explicitly illustrated in FIG. 9, it will be understood that the translucent nature of 3D point clouds may serve as another advantage of 3D point cloud representations over other more opaque object representations (such as volumetric models) for applications in which several potentially-overlapping objects are simultaneously presented. For example, if one practitioner occludes another by occupying space in front of the other practitioner from the viewpoint of the user during the medical procedure, the user may be able to at least partially see through the occluding practitioner to the occluded practitioner due to the translucent nature of the 3D point cloud representation. Moreover, if two practitioners happen to co-occupy the same space during the medical procedure, system 100 is not forced to prioritize one over the other or determine which practitioner to show in that space. Rather, due to the nature of point clouds, 3D points representative of both practitioners can be rendered at that same point and the viewer will be presented with an extra dense (and perhaps multicolored) grouping of points at this area that is intuitively understood to equally represent both practitioners. In some examples, one 3D point cloud may be manually or automatically darkened or lightened, or made to have larger or smaller points, etc., than the other 3D point cloud to help emphasize one 3D point cloud over the other and thereby improve the user's visibility.

As has been described, one way that extended reality content may be made useful and applicable (e.g., particularly for postoperative applications such as have been described) is by organizing 3D point clouds from different medical procedures into a searchable library that may be referenced by practitioners in training, by practitioners preparing for a particular type of medical procedure, by auditors or others analyzing and assessing the quality of different procedures, and/or by other users who may have an interest in viewing such content. For example, in certain implementations, system 100 may manage a library of 3D point cloud data that includes at least 1) data representative of a 3D point cloud depicting practitioner activity associated with a first medical procedure, and 2) additional data representative of a plurality of additional 3D point clouds depicting practitioner activity associated with a plurality of additional medical procedures. Such a library may be searchable to identify particular 3D point clouds represented in the 3D point cloud data based on criteria designated by the user.

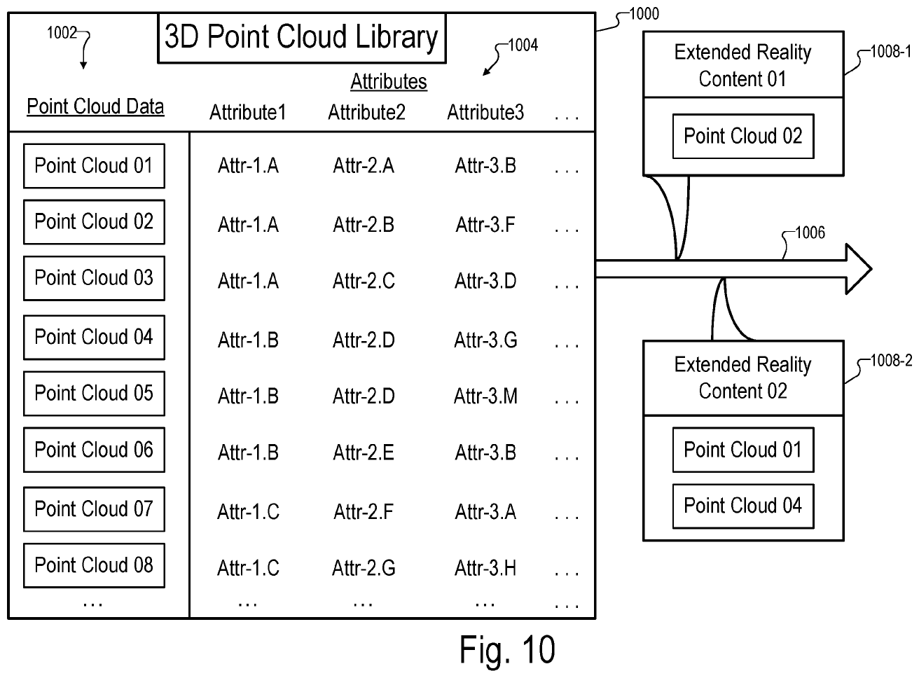

To illustrate, FIG. 10 shows an illustrative 3D point cloud library 1000 used to generate illustrative extended reality content in accordance with principles described herein. As shown, point cloud data 1002 for a plurality of different point clouds (e.g., “Point Cloud 01” through “Point Cloud 08” and additional 3D point clouds represented by an ellipsis at the bottom of the list) may be organized within 3D point cloud library 1000 based on a plurality of attributes 1004 (e.g., “Attribute1”, “Attribute2”, “Attribute3”, and/or other attributes indicated by the ellipsis) that characterize each 3D point cloud. As shown, the particular attributes characterizing the 3D point clouds for which point cloud data 1002 is available are denoted as “Attr-X.Y”, where ‘X’ represents the type of attribute indicated (corresponding to the column in the library representation shown) and where ‘Y’ represents a value for that type of attribute. The search criteria mentioned above may make use of these attributes and allow a user to find and identify 3D point clouds with specifically desired attributes. For example, attributes 1004 may include attributes such as basic information about the medical procedure (e.g., what date the procedure was performed, what time the procedure was performed, who performed various roles during the procedure, what hospital and/or operating room the procedure was performed in, etc.); a medical procedure type (e.g., what type of surgery is represented by the 3D point cloud, what part of the body was being operated on, etc.); an event type (e.g., what types of anomalies were encountered during the medical procedure, etc.); and/or any other attributes characterizing one or more of the 3D point cloud as may serve a particular implementation.

In some implementations, each 3D point cloud for which point cloud data 1002 is available may represent an entire medical procedure from start to finish. In other implementations, some or all of the 3D point clouds represented in the point cloud data 1002 may instead be relevant portions of the medical procedure that are much shorter than the entire procedure would be. For example, as mentioned above, a curator managing the library may identify interesting or important moments in various medical procedures and compile the library with “clips” configured to exhibit these moments in productive ways described herein.

When accessed by a user, 3D point cloud library 1000 may provide output 1006 that is extended reality content incorporating one or more of the 3D point clouds that has been requested (e.g., 3D point clouds represented by point cloud data 1002 that is selected based on criteria associated with attributes 1004). Two examples of extended reality content 1008 are shown in FIG. 10. A first instance of extended reality content 1008-1 (“Extended Reality Content 01”) is shown as an example where a user has selected a single 3D point cloud (“Point Cloud 02” in this example) to experience in the extended reality content. In another example, a second instance of extended reality content 1008-2 is shown as an example where the user has selected multiple 3D point clouds (“Point Cloud 01” and “Point Cloud 04” in this example) to experience them simultaneously in the extended reality content as described above in relation to the example of FIG. 9. Other 3D point clouds, 3D volumetric models, other types of content, and combinations thereof may also be incorporated into output 1006 as directed or requested by the user in certain implementations.

Along with a library of 3D point clouds and/or extended reality content such as illustrated in FIG. 10, other snapshots, video clips, rendered portions of extended reality experiences, and/or additional related content could be captured, tagged, bookmarked, and otherwise maintained and organized for future access and/or sharing within relevant communities (e.g., of practitioners, of students, etc.). For example, content depicting particularly optimal or efficient behaviors (i.e., good examples) and/or particularly deficient or inefficient behaviors (i.e., bad examples) may be managed in a content library that could be provided to and accessed by communities seeking to learn about and review medical procedures for various purposes described herein. In some examples, content in such libraries may be tagged by a community of users (e.g., generating tags when viewing the content). Additionally or alternatively, the content may be automatically tagged by systems such as medical system 300 to indicate particular events or components of the medical procedure (e.g., instrument exchange, docking, cart rollup, etc.).

In certain embodiments, one or more of the processes described herein may be implemented at least in part as instructions embodied in a non-transitory computer-readable medium and executable by one or more computing devices. In general, a processor (e.g., a microprocessor) receives instructions, from a non-transitory computer-readable medium, (e.g., a memory, etc.), and executes those instructions, thereby performing one or more processes, including one or more of the processes described herein. Such instructions may be stored and/or transmitted using any of a variety of known computer-readable media.

A computer-readable medium (also referred to as a processor-readable medium) includes any non-transitory medium that participates in providing data (e.g., instructions) that may be read by a computer (e.g., by a processor of a computer). Such a medium may take many forms, including, but not limited to, non-volatile media, and/or volatile media. Non-volatile media may include, for example, optical or magnetic disks and other persistent memory. Volatile media may include, for example, dynamic random access memory (DRAM), which typically constitutes a main memory. Common forms of computer-readable media include, for example, a disk, hard disk, magnetic tape, any other magnetic medium, a compact disc read-only memory (CD-ROM), a digital video disc (DVD), any other optical medium, random access memory (RAM), programmable read-only memory (PROM), electrically erasable programmable read-only memory (EPROM), FLASH-EEPROM, any other memory chip or cartridge, or any other tangible medium from which a computer can read.

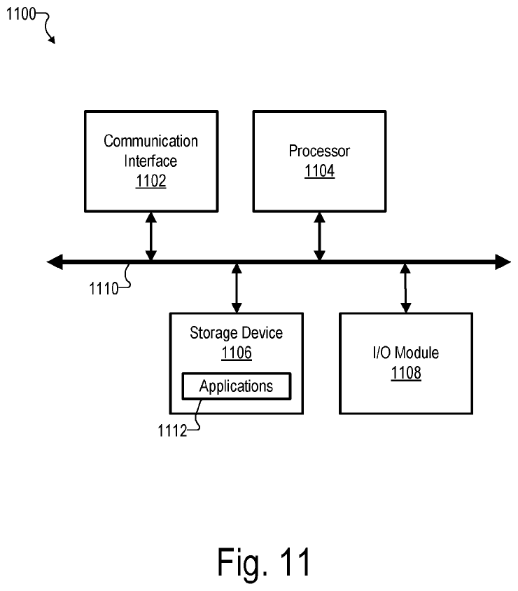

FIG. 11 shows an illustrative computing system 1100 that may be specifically configured to perform one or more of the processes described herein. For example, computing system 1100 may include or implement (or partially implement) a medical procedure visualization system such as system 100, certain components of a medical system such as medical system 300, a depth data capture system such as depth data capture system 402, an extended reality presentation device such as any of devices 410, and/or any other computing systems or devices described herein.

As shown in FIG. 11, computing system 1100 may include a communication interface 1102, a processor 1104, a storage device 1106, and an input/output (I/O) module 1108 communicatively connected via a communication infrastructure 1110. While an illustrative computing system 1100 is shown in FIG. 11, the components illustrated in FIG. 11 are not intended to be limiting. Additional or alternative components may be used in other embodiments. Components of computing system 1100 shown in FIG. 11 will now be described in additional detail.

Communication interface 1102 may be configured to communicate with one or more computing devices. Examples of communication interface 1102 include, without limitation, a wired network interface (such as a network interface card), a wireless network interface (such as a wireless network interface card), a modem, an audio/video connection, and any other suitable interface.

Processor 1104 generally represents any type or form of processing unit capable of processing data or interpreting, executing, and/or directing execution of one or more of the instructions, processes, and/or operations described herein. Processor 1104 may direct execution of operations in accordance with one or more applications 1112 or other computer-executable instructions such as may be stored in storage device 1106 or another computer-readable medium.

Storage device 1106 may include one or more data storage media, devices, or configurations and may employ any type, form, and combination of data storage media and/or device. For example, storage device 1106 may include, but is not limited to, a hard drive, network drive, flash drive, magnetic disc, optical disc, RAM, dynamic RAM, other non-volatile and/or volatile data storage units, or a combination or sub-combination thereof. Electronic data, including data described herein, may be temporarily and/or permanently stored in storage device 1106. For example, data representative of one or more executable applications 1112 configured to direct processor 1104 to perform any of the operations described herein may be stored within storage device 1106. In some examples, data may be arranged in one or more databases residing within storage device 1106.

I/O module 1108 may include one or more I/O modules configured to receive user input and provide user output. One or more I/O modules may be used to receive input for a single virtual experience. I/O module 1108 may include any hardware, firmware, software, or combination thereof supportive of input and output capabilities. For example, I/O module 1108 may include hardware and/or software for capturing user input, including, but not limited to, a keyboard or keypad, a touchscreen component (e.g., touchscreen display), a receiver (e.g., an RF or infrared receiver), motion sensors, and/or one or more input buttons.

I/O module 1108 may include one or more devices for presenting output to a user, including, but not limited to, a graphics engine, a display (e.g., a display screen), one or more output drivers (e.g., display drivers), one or more audio speakers, and one or more audio drivers. In certain embodiments, I/O module 1108 is configured to provide graphical data to a display for presentation to a user. The graphical data may be representative of one or more graphical user interfaces and/or any other graphical content as may serve a particular implementation.

In some examples, any of the facilities described herein may be implemented by or within one or more components of computing system 1100. For example, one or more applications 1112 residing within storage device 1106 may be configured to direct processor 1104 to perform one or more processes or functions associated with processor 104 of system 100. Likewise, memory 102 of system 100 may be implemented by or within storage device 1106.

In the preceding description, various illustrative embodiments have been described with reference to the accompanying drawings. It will, however, be evident that various modifications and changes may be made thereto, and additional embodiments may be implemented, without departing from the scope of the invention as set forth in the claims that follow. For example, certain features of one embodiment described herein may be combined with or substituted for features of another embodiment described herein. The description and drawings are accordingly to be regarded in an illustrative rather than a restrictive sense.

1. A system comprising:
one or more processors; and
memory storing executable instructions that, when executed by the one or more processors, cause the system to:
access depth data captured by one or more depth capture devices during a medical procedure, the depth data representative of practitioner activity associated with the medical procedure; and
render, based on the depth data, extended reality content for presentation to a user, the extended reality content including a 3D point cloud depicting the practitioner activity associated with the medical procedure.

2. The system of claim 1, wherein:
the depth data is captured by a plurality of depth capture devices arranged to have different vantage points at a site where the medical procedure is performed; and
the extended reality content is rendered to show the 3D point cloud to the user from a particular viewpoint at the site where the medical procedure is performed.

3. The system of claim 2, wherein:
the particular viewpoint is a static viewpoint tethered to a static location at the site during the medical procedure; and
the static viewpoint is distinct from the different vantage points of the plurality of depth capture devices arranged at the site.

4. The system of claim 2, wherein the particular viewpoint is a dynamic viewpoint tethered to a practitioner moving about at the site during the medical procedure.

5. The system of claim 2, wherein the particular viewpoint is selected, by a machine learning process or a human curator, as part of a creation of a library of 3D point cloud data configured to demonstrate a variety of medical procedures including the medical procedure.

6. (canceled)

7. The system of claim 1, wherein the extended reality content rendered based on the depth data is hybrid content that includes the 3D point cloud and a volumetric model representing an object at a site where the medical procedure is performed.

8. The system of claim 1, wherein:
the instructions, when executed by the one or more processors, further cause the system to access additional depth data captured during an additional medical procedure distinct from the medical procedure, the additional depth data representative of practitioner activity associated with the additional medical procedure; and
the extended reality content is further rendered, based on the additional depth data, to include an additional 3D point cloud depicting the practitioner activity associated with the additional medical procedure.

9-13. (canceled)

14. The system of claim 1, wherein the rendering of the extended reality content is performed in real time during the medical procedure for presentation to the user as an intraoperative extended reality experience.

15. The system of claim 1, wherein the rendering of the extended reality content is performed after the medical procedure is complete for presentation to the user as a postoperative extended reality experience.

16. The system of claim 1, wherein the instructions, when executed by the one or more processors, further cause the system to direct the presentation of the extended reality content to the user while the user is physically remote from a site where the medical procedure is performed.

17. The system of claim 1, wherein:
the instructions, when executed by the one or more processors, cause the system to manage a library of 3D point cloud data including:
data representative of the 3D point cloud depicting the practitioner activity associated with the medical procedure, and
additional data representative of a plurality of additional 3D point clouds depicting practitioner activity associated with a plurality of additional medical procedures; and
the library is searchable to identify particular 3D point clouds represented in the 3D point cloud data based on criteria designated by the user.

18. The system of claim 1, wherein the instructions, when executed by the one or more processors, further cause the system to:
link the extended reality content to an additional type of content depicting another aspect of the medical procedure occurring concurrently with the practitioner activity; and
provide the user with an interactive presentation of the medical procedure that allows the user dynamic control to view the extended reality content and the additional type of content.

19. The system of claim 18, wherein:
the instructions, when executed by the one or more processors, further cause the system to access sensor data captured by one or more sensors during the medical procedure; and
the additional type of content is generated based on the sensor data.

20. The system of claim 1, wherein the rendering of the extended reality content includes visually differentiating a first practitioner represented by the 3D point cloud from a second practitioner represented by the 3D point cloud by using a first color for points representing the first practitioner and using a second color different from the first color for points representing the second practitioner.

21. The system of claim 1, wherein the rendering of the extended reality content includes visually differentiating a first practitioner represented by the 3D point cloud from a second practitioner represented by the 3D point cloud by using a first label for the first practitioner and using a second label different from the first label for the second practitioner.

22. The system of claim 1, wherein the rendering of the extended reality content includes visually highlighting a first aspect of the 3D point cloud to emphasize the first aspect over a second aspect of the 3D point cloud by using a first color for points representing the first aspect and using a second color different from the first color for points representing the second aspect.

23. The system of claim 1, wherein:
the 3D point cloud depicting the practitioner activity includes a plurality of discrete sub-clouds representing individual people and objects associated with the medical procedure; and
the rendering of the extended reality content includes visually indicating interaction between different discrete sub-clouds by using a first color for adjacent or overlapping points from different discrete sub-clouds and using a second color for other points not adjacent or overlapping with points from different discrete sub-cloud.

24. The system of claim 1, wherein:
the extended reality content further includes sound configured to be presented together with the 3D point cloud depicting the practitioner activity associated with the medical procedure; and
the sound is recorded during the medical procedure or added to the extended reality content as an augmentation after the medical procedure is complete.

25. A method comprising:
accessing depth data captured by one or more depth capture devices during a medical procedure, the depth data representative of practitioner activity associated with the medical procedure; and
rendering, based on the depth data, extended reality content for presentation to a user, the extended reality content including a 3D point cloud depicting the practitioner activity associated with the medical procedure.

26-33. (canceled)

34. A non-transitory computer-readable medium storing instructions that, when executed, direct a processor of a computing device to:
access depth data captured by one or more depth capture devices during a medical procedure, the depth data representative of practitioner activity associated with the medical procedure; and
render, based on the depth data, extended reality content for presentation to a user, the extended reality content including a 3D point cloud depicting the practitioner activity associated with the medical procedure.

35-39. (canceled)
